(19) United States
(12) Patent Application Publication
(10) Pub. No.: US 2011/0117515 A1
(43) Pub. Date: May 19, 2011

(54) Device and method for tooth and gum cleaning for pets using a pen delivery and tooth gel

(76) Inventor: Jennifer Jablow, New York, NY (US)

(21) Appl. No.: 12/925,910

(22) Filed: Nov. 2, 2010

Related U.S. Application Data
(60) Provisional application No. 61/280,459, filed on Nov. 3, 2009.

(51) Int. Cl. A61D 5/00; A61K 8/67; A61Q 11/00

(52) U.S. Cl. 433/1; 424/49

(57) ABSTRACT

A device and method for tooth and gum cleaning in pets with a novel pen delivery and tooth gel.

### RELATED APPLICATIONS

This application is based on provisional patent application Ser. No. 61/280,459 filed Nov. 3, 2009.

### FIELD OF THE INVENTION

The present invention relates to a device and method for tooth and gum cleaning in pets with a novel pen delivery and tooth gel.

### BACKGROUND OF THE INVENTION

The devices on the market for cleaning pets' teeth comprise toothpastes and toothbrushes, rinses, wipes, or drinking additives. These devices do not offer a precise application to the tooth surface. None of the prior art devices provide the advantages of the present invention.

### SUMMARY OF THE INVENTION

The present invention provides a novel delivery of a tooth gel in a device that also has the cleaning surface attached, which allows for easy portability and ease of more precise application to the tooth surface.

It is an object of the present invention for the tooth gel to be a no rinse treatment that is low to no foaming which keeps the surrounding mouth surface clean since there is no expectorating.

It is an object of the present invention for the gel to contain chemical and mechanical properties to remove tartar, debris from tooth surface, sooth gum irritation, deliver gum healing vitamins, and freshen breath. It is an object of the present invention for the tip of the pen to act as a mechanical removal device for removal of debris from the tooth surface.

The present invention relates to a pen device that delivers a tooth gel that cleans a pet's teeth. It is an object of the present invention for the pen device to comprise an angled felt tip, silicone tip, silicone bristles, nylon bristles, nylon and polyester blend bristles, any polymer of plastic, a sponge, or a brush end with orifice to deliver a tooth gel to the pet tooth and gum surface. It is an object of the present invention for the pen device to hold tooth gel in a weight of approximately 0.01 ml to about 18 ml. It is an object of the present invention for the pen device to deliver the gel to the pet tooth surface.

It is an object of the present invention for the pen device to be made of a material comprising a plastic, polypropylene, silicon, and/or surlyn or any combination thereof. It is an object of the present invention for the device to be in the shape of a pen or tube, or a combination thereof. It is an object of the present invention for the device to comprise a felt, sponge, nylon, nylon polyester or silicone tip or any combination thereof.

The present invention relates to a novel tooth gel that delivers cleansing properties both chemically and mechanically to the tooth and gum surface. It is an object of the present invention for the tooth gel to comprise ingredients that are of human grade, safe to use on humans.

The present invention relates to a novel tooth gel comprising a carrier vehicle, an antibacterial agent, anti halitosis agents, an abrasive, and vitamins beneficial to dental health. It is an object of the present invention for the carrier vehicle to comprise glycerin, propylene glycol USP, water, or any combination in the percentage range of about 1 to 80% by weight.

It is an object of the present invention for the antibacterial agent to comprise cetylpyrridium chloride (CPC), chlorhexidine gluconate or any combination of the two in percentages of approximately 0.0025 to about 10% by weight.

It is an object of the present invention for the anti halitosis agent to comprise zinc gluconate, chlorophyll, or any combination thereof in percentage of approximately 0.0025 to about 10% by weight.

It is an object of the present invention for the abrasive to comprise sodium bicarbonate, hydrated silica, titanium dioxide, and or any combination, in a percentage from about 1% to about 50% by weight.

It is an object of the present invention for the tooth gel to comprise a flavor. It is an object of the present invention for the flavor to comprise, vanilla, peanut butter, mint, malt, or any combination in a percentage of approximately 0.1% to about 30% by weight.

It is an object of the present invention for the tooth gel to comprise Vitamins beneficial to dental health. It is an object of the present invention for the vitamins to comprise vitamin C (ascorbic acid) and Co Enzyme Q 10 or a combination thereof in a percentage of approximately 0.1% to about 20% by weight.

It is an object of the present invention for the tooth gel to comprise a pet safe sweetener. It is an object of the present invention for the pet safe sweetener to comprise sorbitol in a percentage of approximately 0.1% to about 30% by weight.

It is an object of the present invention for the tooth gel to have a viscosity of approximately 2,000 centipoise to 100,000 centipoise. It is an object of the present invention for the tooth gel to have a pH of approximately 4.5 to about 7.5.

It is an object of the present invention for the gel to comprise a thickener.

It is an object of the present invention for the thickener to comprise xanthan gum or any pet safe thickener.

It is an object of the present invention for the product to remove any stain product that may adhere to the tooth by the physical silica abrasion (very gentle), baking soda, dicalcium dehydrate or any combination thereof.

It is an object of the present invention for the user to express the gel through the tube to the felt tip and use a circular rubbing motion on the tooth surface. It is an object of the present invention for the composition to be used on all tooth buccal surfaces or just on specific teeth.

It is an object of the present invention for the pet to include a dog, or cat, or any animal that has teeth that need cleaning.

It is an object of the present invention for the dispensing pen to be similar to one's presently on the market for lip gloss or human dental whiteners.

### BRIEF DESCRIPTION OF THE DRAWINGS

FIG. 1 shows a side view of an embodiment of the pen body.

FIG. 2 shows a side view of an embodiment of the pen body.

FIG. 3 shows a side view of an embodiment of the pen body.

FIG. 4 shows a side showing the pen body and the tip on the pen.

FIG. 5 shows a side showing the pen body and the tip on the pen.

FIG. 6 shows a side showing the pen body and the tip on the pen.

FIG. 7 shows a side showing the pen body and the tip on the pen.

FIGS. 8 and 9 show the pen tips associated with FIG. 4 of the present invention.

FIGS. 10 and 11 show the pen tips associated with FIG. 5 of the present invention.

FIGS. 12 and 13 show the pen tips associated with FIG. 7.

### DETAILED DESCRIPTION OF THE INVENTION

The present invention relates to a pen device that delivers tooth gel to clean pets' teeth. The pen device comprises an angled felt tip, silicone tip, silicone bristles, nylon bristles, nylon and polyester blend bristles, any polymer of plastic, a sponge, or a brush end with an orifice to deliver tooth gel to pet tooth and gum surface. In a preferred embodiment, the device holds tooth gel with a volume of approximately 0.01 ml to about 18 ml. In a preferred embodiment, the tip of the device mechanically removes tartar from tooth surface.

In an embodiment, the pen device comprises a material comprising polypropylene, plastic, silicon, and/or surlyn or any combination thereof. In an embodiment, the device is in the shape of a pen or tube with felt, silicone, plastic, nylon, nylon/polyester or sponge tip.

The tooth gel delivers cleansing properties both chemically and mechanically to tooth and gum surfaces of a pet. The gel has ingredients which are of Human Grade, safe to use on humans.

The gel comprises a carrier vehicle, an antibacterial agent, anti halitosis agents, an abrasive and vitamins beneficial to dental health.

In an embodiment, the carrier vehicle comprises glycerin, propylene glycol USP, water or any combination in the percentage range of about 0.1 to about 80% by weight.

In an embodiment, the antibacterial agent comprises cetylpyrridium chloride (CPC), Chlorhexidine gluconate or any combination of two in percentages of about 0.0025% to about 10% by weight.

In an embodiment, the anti halitosis agents comprise zinc gluconate, chlorophyll or any combination of in percentages of about 0.0025% to about 10% by weight.

The abrasive of the gel mechanically removes debris from a pets tooth. In an embodiment, the abrasive material comprises sodium bicarbonate, hydrated silica, titanium dioxide or any combination of in a percentage from about 0.1% to about 50% by weight.

In an embodiment, the gel comprises a flavoring agent. In an embodiment, the flavoring agent comprises vanilla, peanut butter, mint, malt or any combination in a percentage from about 0.1% to 30% by weight.

In an embodiment, the gel comprises tooth gel that contains vitamins beneficial for the gum tissue. In an embodiment, the vitamins comprise Vitamin C (ascorbic acid) or Co Enzyme Q 10 or any combination thereof in percentage of about 0.1% to about 20% by weight.

In an embodiment, the gel further comprises a pet safe sweetener. In an embodiment, sweetener comprises sorbitol in a percentage of about 0.1% to about 30% by weight.

In an embodiment, the gel has a viscosity of approximately 2,000 centipoise to about 100,000 centipoise. In an embodiment, the gel has a pH of about 4.5 to about 7.5. In an embodiment, the gel further comprises a thickener. In an embodiment, the thickener comprises xanthan gum, or any pet safe thickener.

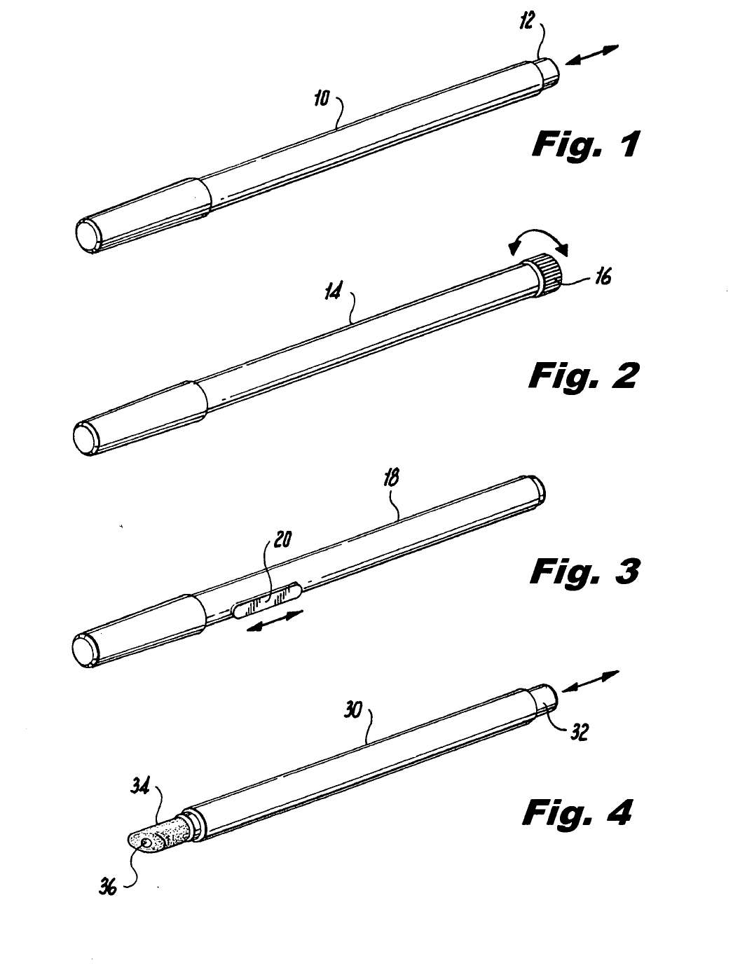

FIG. 1 shows a pen body 10 having a click button 12 for distributing the gel in the pen body 10.

FIG. 2 shows a pen body 14 having a twist button 16 for distributing the gel located in the pen body 14.

FIG. 3 shows the pen body 18 having a side click button 20 for distributing the gel located in the pen body 18.

FIG. 4 shows the pen body 30 having a click button 32 and a pen tip 34. The pen tip 34 has an orifice 36 from which the gel is distributed from the pen body 30. The tip 34 is made from sponge, felt, silicon, plastic or surlyn or any combination thereof.

FIG. 5 shows the pen body 40 having a click button 42 and a pen tip 44. The pen tip has bristles 46 and an orifice 48. The bristles 46 comprise silicone, nylon, nylon/polyester or sponge tip.

FIG. 6 shows a pen body 50 having a click button 52 and a pen tip 54. The pen tip has bristles 56 and an orifice 58.

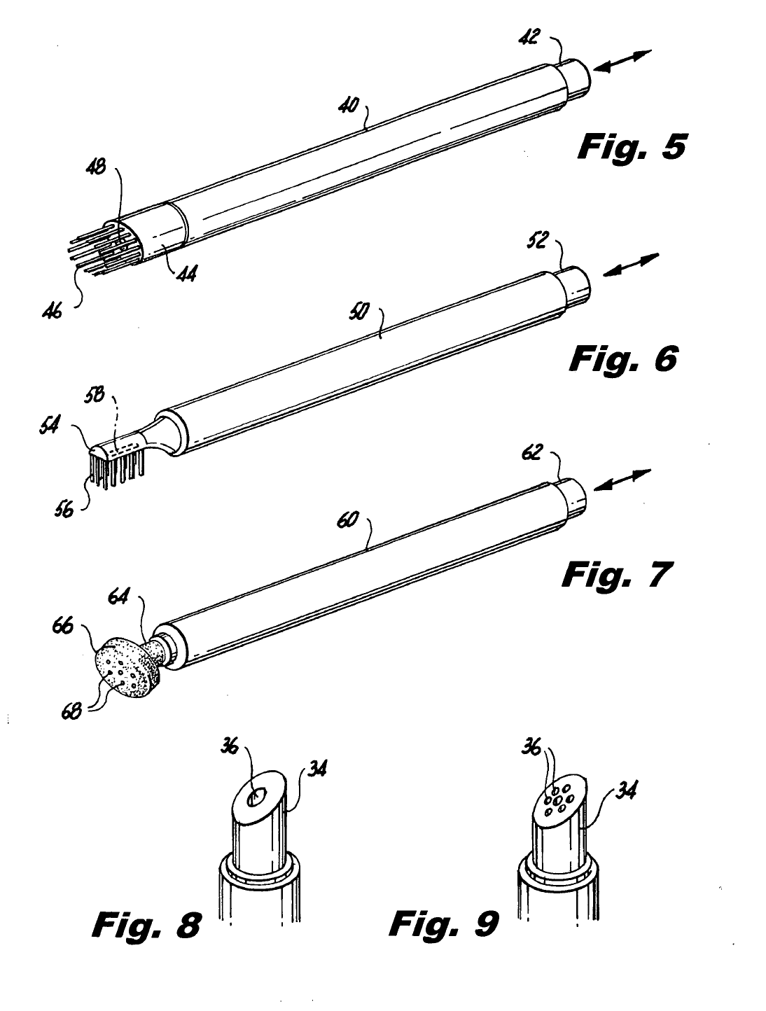

FIG. 7 shows a pen body 60 having a click button 62, a pen tip 64 comprising a sponge 66 having a single or multiple orifices 68.

FIG. 8 shows the pen tip 34 having an orifice 36 which is a single orifice.

FIG. 9 shows the pen tip 34 of FIG. 4 having multiple orifices 36.

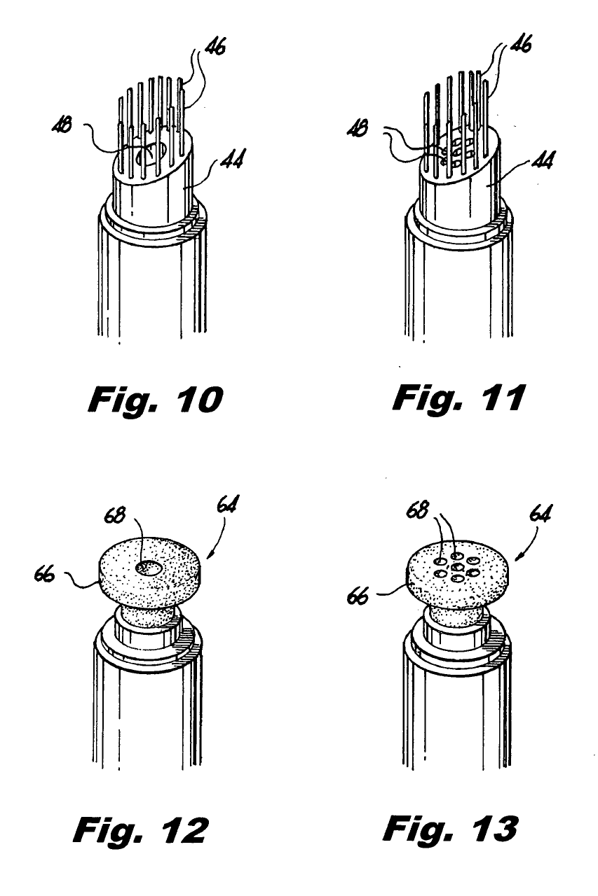

FIG. 10 shows pen tip 44 having bristles 46 and a single orifice 48.

FIG. 11 shows pen tip 44 having bristles 46 and multiple orifices 48.

FIG. 12 shows a pen tip 64 being made of a sponge 66 having a single orifice 68.

FIG. 13 shows a pen tip 64 being made of a sponge material 66 having multiple orifices 68.

1. A pen device that delivers tooth gel to clean pets teeth.

2. The pen device of claim 1 wherein said device comprises an angled felt tip, silicone tip, silicone bristles, nylon bristles, nylon/polyester, sponge, or a brush end with an orifice to deliver tooth gel to pet tooth and gum surface.

3. The pen device of claim 1 wherein said device holds tooth gel with a weight of approximately 0.01 ml to about 18 ml.

4. The pen device of claim 1 wherein a tip of said device mechanically removes tartar from tooth surface.

5. The pen device of claim 1 wherein said device comprises polypropylene, any form of plastic, or surlyn, or any combination thereof.

6. The pen device of claim 2 wherein said device is in shape of a pen or tube with felt, sponge, nylon, nylon polyester, or silicone tip or any combination thereof.

7. A tooth gel that delivers cleansing properties both chemically and mechanically to tooth and gum surfaces of a pet.

8. The gel of claim 7 wherein ingredients of said gel are of human grade, safe to use on humans.

9. The gel of claim 7 wherein said gel comprises a carrier vehicle, an antibacterial agent, anti halitosis agents, an abrasive and vitamins beneficial to dental health.

10. The gel of claim 9 wherein said carrier vehicle comprises glycerin, propylene glycol USP, water or any combination in the percentage range of about 0.1% to about 80% by weight.

11. The gel of claim 9 wherein said antibacterial agent comprises cetylpyrridium chloride (CPC), Chlorhexidine gluconate or any combination of two in percentages of about 00025% to about 10% by weight.

12. The gel of claim 9 wherein said anti halitosis agents comprise zinc gluconate, chlorophyll or any combination of in percentages of about 0.0025% to about 10% by weight.

13. The gel of claim 9 wherein said abrasive mechanically removes debris from a pets tooth.

14. The gel of claim 13 wherein said abrasive material comprises sodium bicarbonate, hydrated silica, titanium dioxide or any combination of in a percentage from about 0.1% to about 50% by weight.

15. The gel of claim 9 further containing a flavoring agent which comprises vanilla, peanut butter, mint, malt or any combination of in a percentage from about 0.1% to 30% by weight.

16. The gel of claim 9 further comprising a tooth gel that contains vitamins beneficial for the gum tissue.

17. The gel of claim 17 wherein said vitamins comprise vitamin C (ascorbic acid) or Co Enzyme Q 10 or any combination thereof in percentage of about 0.1% to about 20% by weight.

18. The gel of claim 9 further comprising a pet safe sweetener in a percentage of about 0.1% to about 30% by weight.

19. The gel of claim 9 wherein said gel has a viscosity of approximately 2,000 centipoise to about 100,000 centipoise.

20. The gel of claim 9 wherein said gel has a pH of about 4.5 to about 7.5.

21. The gel of claim 9 further comprising a thickener.
